(19) United States
(12) Patent Application Publication
(10) Pub. No.: US 2025/0344677 A1
(43) Pub. Date: Nov. 13, 2025

(54) PIT Tag implanter, implanting needle, and stackable implanting needle tray

(71) Applicant: VODA IQ LLC, Hammett, ID (US)

(72) Inventor: Brian Beckley, Hammett, ID (US)

(21) Appl. No.: 19/203,166

(22) Filed: May 8, 2025

Related U.S. Application Data
(60) Provisional application No. 63/644,405, filed on May 8, 2024.
(60) Provisional application No. 63/744,930, filed on Jan. 14, 2025.

(51) Int. Cl. A01K 61/95; A61M 37/00

(52) U.S. Cl. CPC A01K 61/95; A61M 37/0069; A61M 2250/00

(57) ABSTRACT

A PIT tag implanter that has a gun like implanter for implanting a PIT tag into a target animal or fish. The implanter has a needle attachment end, a needle ejection mechanism, and a PIT tag ejection mechanism to eject the PIT tag from the needle. In use, a needle is affixed to the muzzle of the implanter gun. The tip of the needle is inserted through the skin of a target. The actuator is depressed, causing a push rod to eject the PIT tag from the needle into the target. The needle is withdrawn from the target and a needle ejection mechanism ejects the needle. A new needle pre-loaded with a PIT tag is loaded into the muzzle. The improved gun allows for faster implantation, ejection of the needle, and loading of a new needle than prior devices.

### CROSS-REFERENCE TO RELATED APPLICATIONS

This application claims the benefit of U.S. Provisional Application No. 63/644,405, filed May 8, 2024, and U.S. Provisional Application No. 63/744,930, filed Jan. 15, 2025, the disclosure of each of which is incorporated by reference.

### FIELD OF THE INVENTION

The disclosure generally relates to the field of implantation of passive integrated transponders tags (PIT Tags). Particular embodiments relate to an improved implanting device for implanting a PIT Tag into a carrier, and an improved stackable tray apparatus and system for providing single use needles pre-loaded with PIT tag for implanting of the PIT Tag with a PIT Tag implanter.

### BACKGROUND OF THE INVENTION

Passive Integrated Transponders, or PIT Tags, are tracking tags that are commonly used to monitor movement, survival, and habitat use of wildlife, as well as for tracking and identification of domestic animals. PIT Tags are a glass-encapsulated microchip approximately the size of a rice grain. The microchip is activated when the microchip passes close to a special detecting antenna. The antenna is connected to a computer that records the identity of the Pit Tag in the time that it passed by the antenna. PIT Tags can be attached to the animal, such as in a leg band, or subcutaneously injected into the animal.

Prior art implanting guns typically have a needle attachment/detachment mechanism for attachment and detachment of the needle to the implanting gun, and an implanting mechanism for injecting the PIT Tag from the pre-loaded needle into the animal. The actuation mechanism causes a rod to extend through the barrel of the gun, ejecting the PIT Tag from the needle and into the animal. The tip of the needle is typically beveled for implanting into the abdomen of the smolt, creating a small opening into which the PIT Tag is positioned.

A prevalent use of PIT Tags is for tracking anadromous fish such as salmon and steelhead that migrate from the ocean to spawn in inland rivers. PIT Tags can be inserted into adult fish or juvenile fish, called smolt. PIT Tags are inserted in hatchery reared smolts before the smolts are released into a river or stream. Typically the PIT Tag is injected into the smolt utilizing a specialized implanting device, referred to herein as an “implanting gun.” Through the use of pre loaded needles and an implanting gun a technician can implant PIT Tags into a large number of smolt in a short amount of time. The pre loaded needles typically are provided on a tray having a series of needles, for example fifty or one hundred pre-loaded needles. The technician can rapidly load a needle into the muzzle of the implanting gun, implant the tag into the smolt, and discard the needle, and load a new needle. What is disclosed is an improved PIT Tag implanting gun having an improved loading and implanting mechanism for attaching a needle to the muzzle of the gun.

### SUMMARY

The purpose of the Summary is to enable the public, and especially the scientists, engineers, and practitioners in the art who are not familiar with patent or legal terms or phraseology, to determine quickly from a cursory inspection, the nature and essence of the technical disclosure of the application. The Summary is neither intended to define the inventive concept(s) of the application, which is measured by the claims, nor is it intended to be limiting as to the scope of the inventive concept(s) in any way.

What is disclosed is a handheld passive integrated transponder tag (PIT Tag) gun implanter, an improved implanting needle for use with the gun implanter, an improved tray system for holding implanting needles.

The implanter gun having a handgun shaped housing having a barrel portion and a handle portion. The handle portion being disposed on said housing for being grasped by a hand of a user. The barrel portion extends forward to a muzzle. The gun housing having an opening at the muzzle configured to receive a needle hub.

A barrel is positioned within the barrel portion of the housing. The barrel portion having a needle seat at the muzzle and a hammer end. A push rod is housed at least partially in the barrel. The push rod is configured to slide in the barrel. A push rod actuator causes the push rod to slide in the barrel. A releasable retention hook positioned in said barrel portion of said gun housing and is configured to retain a retention flange of the needle. A retention hook trigger is positioned at the hammer end of the barrel portion of the housing and configured to release the retention hook from said retention flange, allowing the needle to be removed or ejected from the muzzle.

The needle has needle hub releasably positioned in the muzzle against the needle seat of the barrel. The needle has a hollow steel rod comprising a beveled tip, said hollow steel rod extending from said needle hub and said barrel end of said housing, said needle hub comprising a retention flange secured by said retention hook. A passive integrated transponder tag is positioned within the needle. The needle is secured in the muzzle of the gun such that the needle is retained by the releasable retention hook. The push rod actuator is configured to slide the push rod into the needle to eject the PIT Tag from tip of said needle and into the recipient of the PIT Tag. The needle is beveled to facilitate piercing the skin of the recipient of the tag. Preferably the needle has two or more alignment tabs that facilitate quick alignment of the needle in the muzzle.

In the preferred embodiment the needle base has two opposing positioning tabs such that positioning the needle in a first alignment positions the bevel on a first side of the gun, and rotating the needle 180 degrees positions the bevel on the opposite side of the gun. This facilitates use of the gun by left handed persons in one orientation, and right handed persons in the second orientation.

The barrel is configured to slide in the gun housing to accept and eject the needle. In use, the needles are in a tray with the needle hub extending above the tray. The muzzle of the gun is depressed onto the needle. This needle base seats against the needle seat of the muzzle, pushing the barrel toward the opposite end of the barrel section until it reaches an arrest. A spring biases the barrel toward the muzzle, and the arrest stops the barrel from sliding. As the barrel slides, retention hooks secure the needle hub in the muzzle of the gun. Preferably these retention hooks rotate into securing position as the barrel slides toward the back of the gun housing. As the barrel moves forward, the retention hooks rotate away from the needle base, releasing the base.

The spring can be positioned between the end of the barrel and the housing or along the barrel between a barrel spring seat and a housing spring seat. The housing spring seat is stationary, allowing the spring to extend and compress as the barrel slides.

In a preferred embodiment two retention hooks are positioned on opposite sides of the barrel. The retention hooks pivotally connected to the housing such that the retention hooks rotate relative to the housing. In a preferred embodiment each of the retention hooks comprises one of a detent or detent notch, and the barrel comprises the other of said detent or detent notch such that sliding of the barrel forward and back causes the pivotal rotation of the retention hooks.

The arrest can be in the form of a flange on or near the end of the barrel opposite the muzzle. This actuation flange is positioned such that a retention hook trigger has a hook that engages the flange. Depressing the retention hook trigger, preferably a tab, releases the actuation hook from the actuation flange, allowing the spring to bias the barrel forward.

The push rod actuator is positioned in the handle of the gun. In a preferred embodiment this is a trigger that is actuated by a user squeezing his or her fingers toward the handle. In the preferred embodiments the actuator is connected to a lever that is pivotally connected to the housing. Squeezing the actuator causes the rod to move forward, sliding the push rod forward in the barrel. A tension spring biases the actuator back when the user releases pressure on the actuator, causing the push rod to slide back into the barrel.

An improved tray system for delivery of a plurality of needles pre-loaded with PIT Tags is also disclosed. This tray system can utilize a plurality of trays, but for this purpose the interaction of two of the trays is addressed. This is a pair of mating trays with each tray having a tray top having a plurality of needle sheath openings that are configured to receive a needle pre-loaded with a PIT Tag. The needle sheaths extend beneath the tray top. The needle sheaths extending to a needle sheath tip. Each tray has a plurality of storage openings configured to receive the needle sheath tips such that said mating trays are in a stacked arrangement.

In a preferred embodiment one of the needle sheaths and said storage openings has a crush ring configured to deform to increase friction between said needle sheaths and said storage openings.

The preferred embodiment of the needle for use with a PIT Tag implanter gun has a cylindrical hub. This cylindrical hub is mounted in a muzzle of a PIT Tag implanter gun. The hub comprising a cylindrical conduit therethrough. The hub having an external surface having a retention flange and two opposing alignment tabs. The alignment tabs being configured to engage two opposing alignment slots in the PIT Tag implanter gun.

The needle has a hollow tube defining a lumen. The hollow tube, preferably metal such as steel, extends from the cylindrical hub and is coaxial with or continuous with the cylindrical conduit of the cylindrical hub such that a push rod in the implanter gun extends into said needle to expel a PIT Tag from said needle through the tip of the hollow steel tube, said hollow steel tube comprising a bevel. The positioning tabs align the bevel of the steel tube to extend to be orientated to a side of the implanter gun.

Still other features and advantages of the presently disclosed and claimed inventive concept(s) will become readily apparent to those skilled in this art from the following detailed description describing preferred embodiments of the inventive concept(s), simply by way of illustration of the best mode contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature

### BRIEF DESCRIPTION OF DRAWINGS

FIG. 1 is an isometric view of a first embodiment of an implanting gun in an unloaded state.

FIG. 2 is an isometric view of the implanting gun of FIG. 1 in a loaded state with a needle.

FIG. 3 is an isometric view of the gun of FIG. 1 having a body panel of the gun removed to illustrate the internal components of the gun.

FIG. 4 is an exploded view of the gun and needle of FIG. 2 with the needle exploded from the gun and a body panel of the gun removed.

FIG. 5 is a side view of the gun loaded with the needle and the push rod in a loaded position and a body panel of the gun removed.

FIG. 6 is an isometric view of the gun loaded with a needle and the pushrod in the extended position.

FIG. 7 is a side view of the gun with a loaded needle and illustrating the implanting actuator in the depressed position causing the actuation lever to move the push rod forward and eject the PIT tag from the needle.

FIG. 8 is a back view of the implanter gun and needle of FIG. 7 showing section line B-B.

FIG. 9 is a section view along line B-B of FIG. 8.

FIG. 9.1 is front view of a cylindrical needle hub.

FIG. 9.2 is a side view of the cylindrical needle hub of FIG. 9.1

FIG. 9.3 is front view of a needle for use in an implanting gun disclosed herein.

FIG. 9.4 is a section view along line C-C of FIG. 9.3.

FIG. 10 is an isometric view of a needle tray.

FIG. 11 is an isometric view of pair of stacked needle trays.

FIG. 12 is a top view of a needle tray.

FIG. 13 is an end view of the needle tray of FIG. 12.

FIG. 13.1 is a side view of the needle tray of FIG. 12.

FIG. 14 is a top view of the needle tray of FIG. 12 illustrating detail view location of FIG. 14.1.

FIG. 14.1 is a detail view taken at Detail A of FIG. 14.

FIG. 15 is an end view of the needle tray of FIG. 12 illustrating section line C-C of FIG. 16.

FIG. 16 is a section view along line C-C of FIG. 15 and illustrating detail location of FIG. 17.

FIG. 17 is a detail view taken at Detail D of FIG. 16.

FIG. 18 is an end view of the needle tray of FIG. 12 having a series of needles positioned in the needle tray.

FIG. 19 is a section view along line M-M of FIG. 18 and illustrating detail location of FIG. 20.

FIG. 20 is a detail view taken at location O of FIG. 19.

FIG. 21 is an isometric view of a preferred embodiment of an implanter gun with the side gun housing removed and without a needle loaded on the gun and the compression spring extended with the barrel slid toward the muzzle of the gun.

FIG. 22 is a section view of FIG. 21.

FIG. 23 is an isometric view of the implanter gun of FIG. 21 with the side gun housing removed and with a needle loaded on the gun muzzle and the compression spring compressed with the barrel slid toward the butt of the gun.

FIG. 24 is a section view of FIG. 23.

### DETAILED DESCRIPTION OF THE INVENTION

While the presently disclosed inventive concept(s) is susceptible of various modifications and alternative constructions, certain illustrated embodiments thereof have been shown in the drawings and will be described below in detail. It should be understood, however, that there is no intention to limit the inventive concept(s) to the specific form disclosed, but, on the contrary, the presently disclosed and claimed inventive concept(s) is to cover all modifications, alternative constructions, and equivalents falling within the spirit and scope of the inventive concept(s) as defined in the claims.

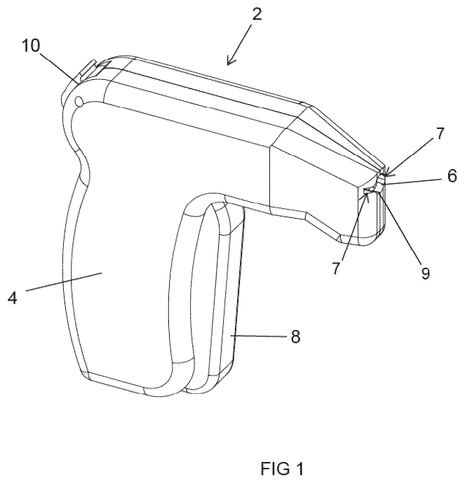

FIG. 1 illustrates an isometric view of the improved gun according to the inventive concepts disclosed herein. The implanting gun has a housing in the shape of a gun (a hand gun in particular), having a barrel portion and a handle portion. The depicted barrel portion has a front end at the muzzle of the gun, and a handle end where the firing hammer typically would be positioned.

FIG. 1 illustrates the improved implanting gun in an unloaded position with the needle release trigger positioned in a released position. The improved gun 2 has a handle 4 and a muzzle 6. The gun is formed by a housing 11 that houses a mechanism for ejecting the PIT Tag, as well as for retaining and ejecting a needle from the muzzle of the gun.

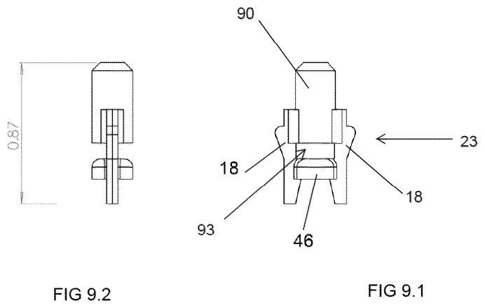

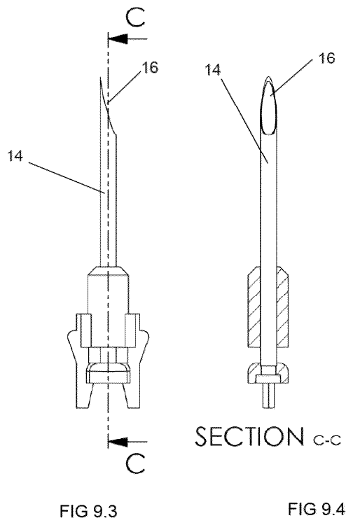

The handle of the implanter gun has a trigger or implanting actuator 8 that is depressed ejecting a PIT Tag from the needle. The muzzle 6 has a circular aperture 9 that is configured for receiving the hub of the needle. The opening has two alignment slots 7 that are configured to receive opposing alignment tabs 18 of the hub of the needle. The alignment tabs facilitate the correct orientation of the needle on the gun such that the bevel of the needle tip is oriented in the correct direction. In the depicted embodiment the flange 46 of the needle hub is in a spaced apart relationship from the rod seat 90 by the two alignment tabs 18. This spaced apart relationship provides a gap 93 (shown in FIG. 9.1) into which a retaining hook engages to secure the needle in the muzzle of the gun. The needle houses a PIT Tag, which is ejected from the needle when the implanting actuator 8 is depressed. Depression of the actuator causes a push rod positioned within the barrel of the gun to slide forward in the barrel and into the needle, propelling the PIT Tag from the end of the needle. Each needle is loaded with a single PIT Tag and is discarded after the PIT tag is injected into the animal.

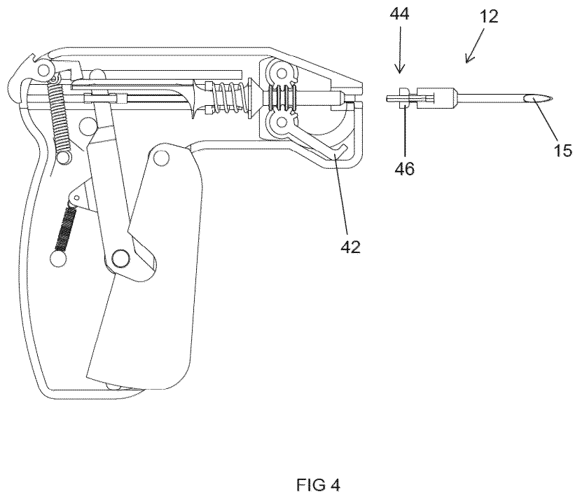

A release trigger or tab 10 is positioned at the opposite end (or handle end) of the barrel from the muzzle where the needle is loaded. The release trigger is pushed from a first position, in which the needle is loaded against the barrel, to a second or depressed position to eject the needle from the muzzle of the gun. The barrel of the gun is biased to the ejected position when the tab 10 is unlatched from the barrel. When the needle is pressed against the barrel, the barrel slides back toward the tab 10. An end of the tab 10 engages the barrel, as shown in FIG. 4, preventing the barrel from being pushed forward. Releasing the tab from the barrel allows a spring to push the barrel forward, ejecting the needle from the muzzle of the gun.

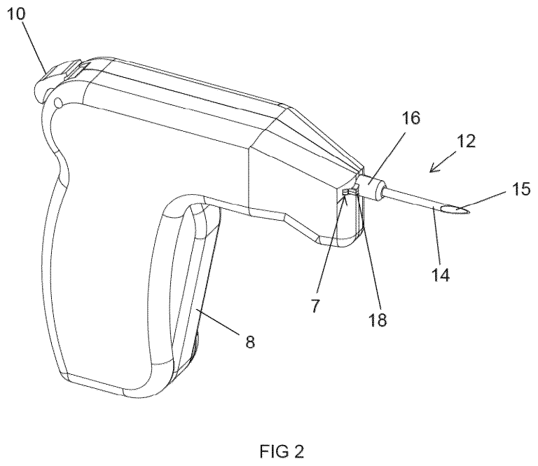

FIG. 2 illustrates the gun with a needle loaded into the muzzle. The needle 12 has a hub 16 and hollow rod 14 extending from the hub to a tip 15. The tip 15 is beveled to facilitate piercing of the tip into the skin of an animal, creating a small incision in the skin of the animal that extends through the skin of the animal. The PIT Tag is then propelled from the lumen of the needle, out the tip of the needle, and into the incision to position the PIT Tag beneath the skin of the animal, or in the case of a smolt, into the abdomen of the fish.

The hub 16 of the needle has two alignment tabs 18 on opposite sides of the hub of the needle. The alignment tabs are inserted into opposing slots 7 formed in the muzzle of the gun. The release tab 10 is positioned in a cocked or loaded position in comparison to the hammer position of FIG. 1.

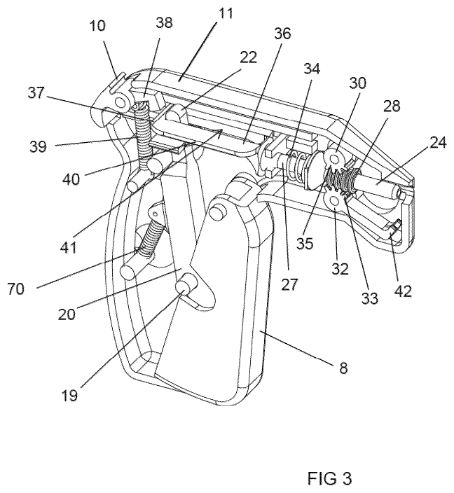

FIG. 3 illustrates an isometric view of the gun of FIGS. 1-2 having a handle cover removed to illustrate the needle loading mechanism and the PIT Tag implanting mechanism. The needle loading mechanism utilizes release trigger 10 that is pivotally connected to the gun housing. The release trigger has a loading hook that is configured for releasable connection to a retaining flange 37 on the barrel. The embodiment of FIGS. 1-9 utilizes a barrel having a split bracket 36 defining a slot to accommodate the push rod actuation lever within the barrel. A tension spring 39 biases the release trigger such that the retaining hook is biased to connect to the retaining tab 37 of the release trigger 10 to the split bracket 36. The split bracket defines a channel in which the actuation lever propels the push rod toward the lumen of the needle.

The barrel of the injector gun of FIGS. 1-9 is formed of a forward barrel 24 and rear barrel 27. The barrel is in connection with the split bracket such that retention of the spit bracket by the tab 10 prevents the barrel from sliding forward. The rear barrel 27 is slideably positioned in positioning bracket 47. The positioning bracket in the depicted embodiment forms a spring seat for the first end of compression spring 34. The second end of the compression spring is positioned against a barrel spring seat connected to the barrel. The compression spring biases the barrel forward, or from left to right in FIG. 3.

The forward barrel has a series of detents 28 in the outer circumference of the barrel that engage with two opposing wheels 30, 32 positioned on opposite sides of the forward barrel. The two opposing wheels have detent notches 33, 35 configured for engagement with the detents in the outer circumference of the barrel. The lower wheel has a needle retention hook 42 that is configured for engagement with the needle retention flange of the needle.

To load a needle onto the gun from a tray of needles, the muzzle of the gun is pressed onto the hub of the needle. The alignment tabs facilitate correct alignment of the needle in the muzzle of the gun. The hub of the needle inserts into the muzzle of the gun against the end of the forward barrel 24 and pushes the barrel toward the rear of the gun. The sliding of the barrel toward the rear of the gun causes the compression spring 34 to compress. The barrel slides toward the rear of the gun until the split bracket 36 reaches the is biased by the extension spring into engagement with the rear flange of the split bracket. This loads the needle into the muzzle of the gun. As the forward barrel 24 is pushed back toward the release tab, the detents on the circumference of the barrel engage the detent notches on the opposing wheels, rotating the wheels toward the actuator tab. This rotation rotates the needle hook 42 into engagement with the retaining flange of the hub of the needle, securing the needle in the muzzle of the gun.

To release a needle from the gun, a user depresses the release tab. Releasing the tab releases the retaining hook 28 from the flange of the split bracket. The compression spring 34 extends, causing the barrel to move forward and away from the rear of the gun. Movement of the barrel forward causes the detents 28 to move toward the muzzle of the gun, causing rotation of the wheels 30, 32, and rotating the hook 42 out of engagement with the hub of the needle, allowing the needle to fall from or be removed from the gun.

The implanting mechanism functions to propel a PIT Tag through the lumen of the needle and into an incision created by the end of the need. The implanting actuator 8 is connected to an actuation lever 20. Depression of the implanting actuator 8 causes the actuation lever to pivot at pivot point 19, causing the end of the actuation lever 22 to travel forward (from left to right in FIG. 3). The actuation lever is positioned between the two opposing arms of the barrel, which forms a channel or guide in the depicted embodiment in the form of a split bracket. A push rod 48 (illustrated in FIGS. 4 and 9) is connected to the actuation lever and positioned within the barrel. When the implanting actuator 8 is depressed, the actuation lever pushes the push rod into the lumen of the needle, propelling the PIT Tag through the lumen 21 of the needle and out the tip of the needle.

FIG. 4 illustrates a side view of the gun of FIG. 3. A needle is shown exploded from the muzzle of the gun illustrating the hub 44 of the needle having a retaining flange. FIG. 5 illustrates the gun of FIGS. 3 and 4 having the needle loaded into the muzzle of the gun. The muzzle of the gun has been pushed onto the hub of the needle, with the alignment tabs 18 of the hub of the needle positioned into the alignment slots of the muzzle of the gun. The retaining flange 46 of the needle is retained by retaining hook 42 of the gun. The forward barrel has pushed in the direction of the hammer, compressing the spring 34 until the flange of the split bracket is engaged by the hammer 38.

FIG. 5 illustrates the actuation lever and push rod in a loaded or cocked position. The actuation lever and first end of the push rod are positioned proximate to the release trigger. Depression of the actuator causes the push rod to slide forward thus ejecting the PIT Tag from the needle.

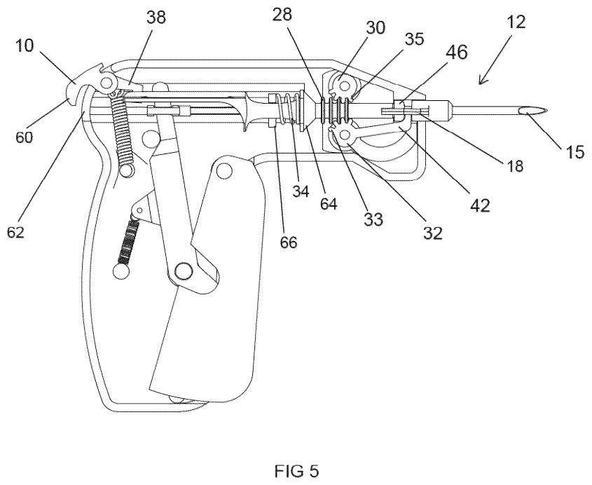

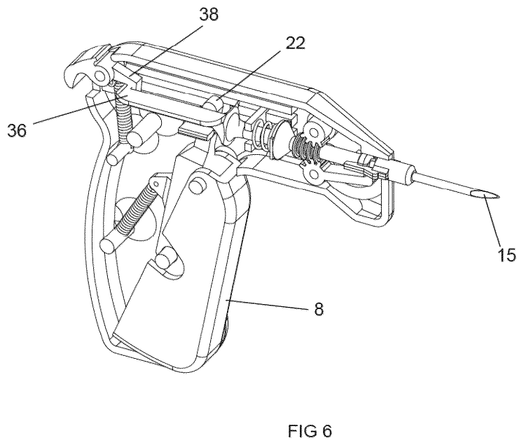

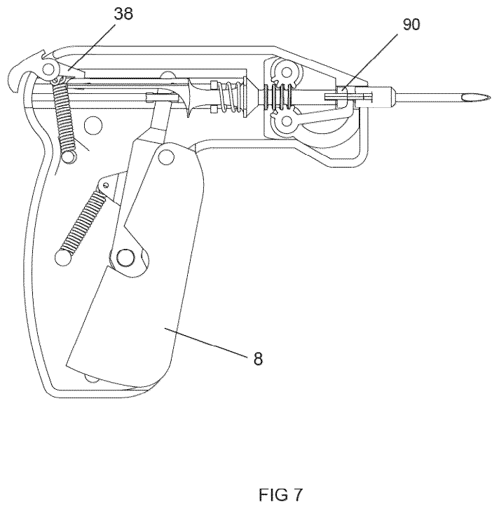

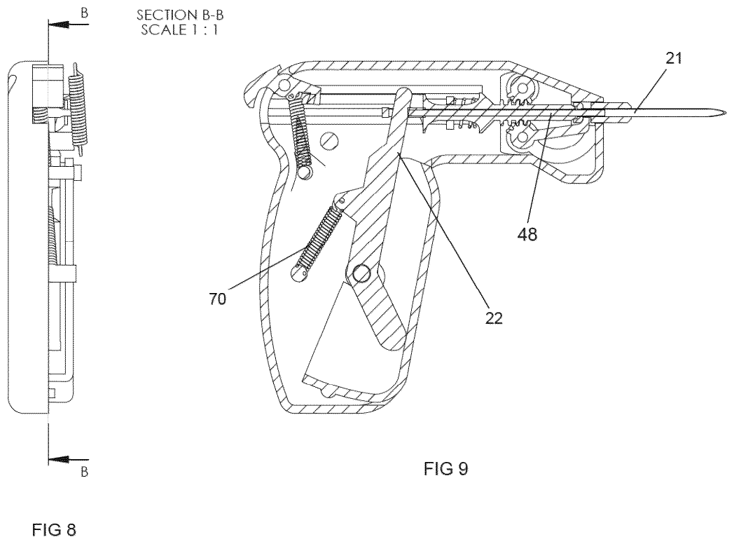

FIG. 6 illustrates the gun of FIGS. 1-5 in which the actuation lever 22 has been actuated causing the PIT Tag to eject from the tip 15 of the needle. The slot of the split bracket allows for the travel of the push rod actuator from the cocked position forward to eject the PIT Tag. FIG. 7 illustrates a side view of the gun and orientation of FIG. 6. FIG. 8 illustrates a back view of the gun of FIG. 7.

FIG. 9 illustrates a section view along section line B-B of FIG. 8. The push rod 48 is illustrated positioned in the barrel of the gun. The tension spring 70 biases the actuation lever 22 toward the handle end of the gun, away from the muzzle of the gun. This biasing action withdraws the push rod 48, which is attached to the actuation lever, into the barrel, allowing for sequential loading of a needle and ejection of the PIT Tag and push rod. The rotation of the actuation lever biases the lever actuator 8 outward to in essence load the actuator, or trigger. A user can then depress the actuator, ejecting the PIT Tag from the next needle that is loaded onto the muzzle of the gun.

FIGS. 9.1-9.5 illustrate a preferred embodiment of a needle for use with the implanting gun of FIGS. 1-9. FIGS. 9.1 and 9.2 illustrate the hub 16 of the needle with the hollow steel rod detached. FIGS. 9.3-9.6 illustrate the needle with the hollow rod attached. The hollow steel rod extends through the rod seat to the retaining flange 46. The bevel 23 in the tip 15 of the needle is oriented in the depicted embodiment toward one of the alignment tabs 18, allowing the bevel to be positioned to one side of the gun, with a one hundred eighty (180) degree rotation of the needle to position the bevel on the opposite side of the gun to align the needle for opposite handed users.

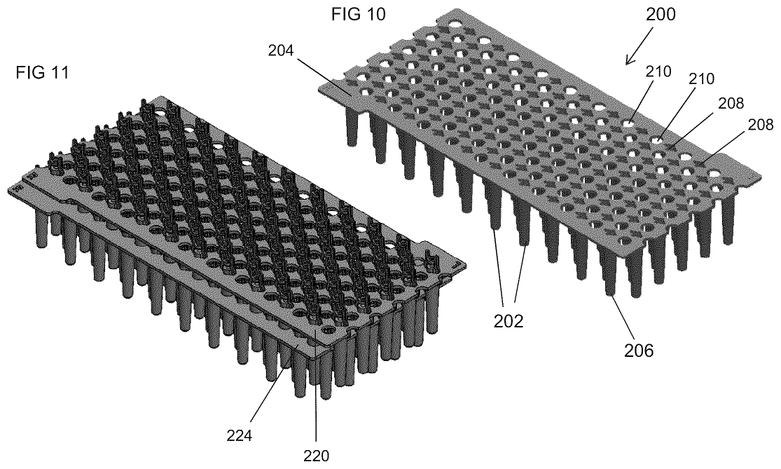

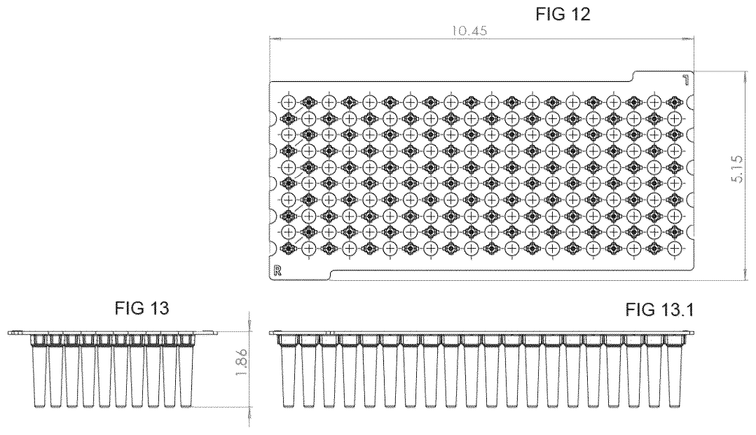

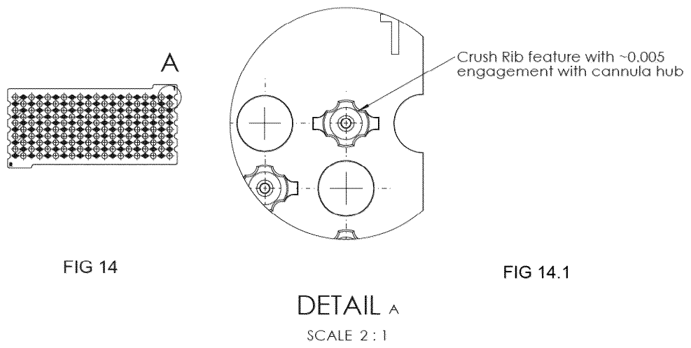

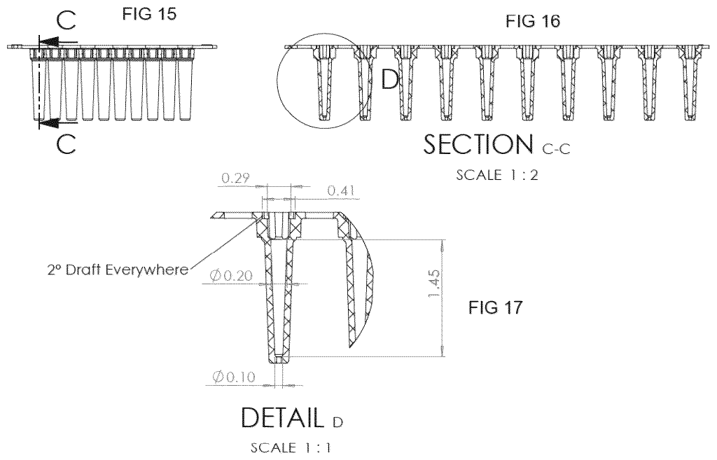

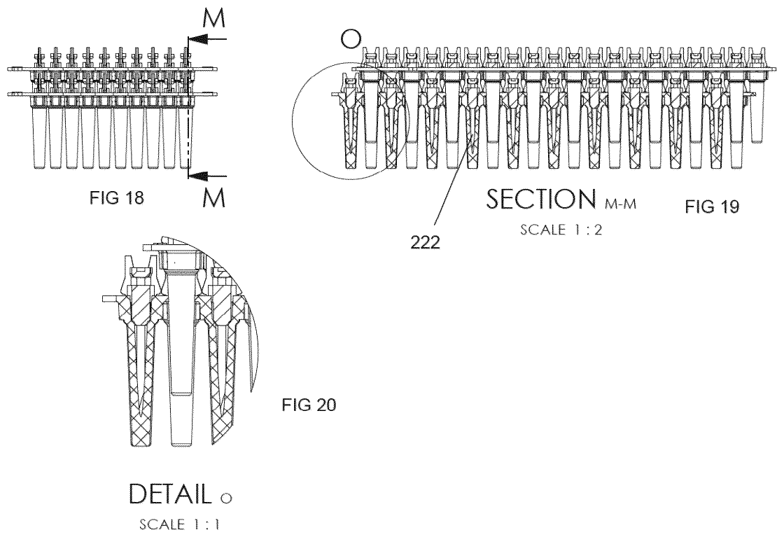

FIGS. 10-20 illustrate an improved tray 200 having an improved tray stacking mechanism. Each of the trays comprises a series of needle sheaths 202 extending beneath a horizontal tray top 204 of the tray. The horizontal tray top has an alternating series of sheath openings 208 to the needle sheaths and stacking openings 210. The stacking openings are configured for insertion therein of the ends or tips 206 of the needle sheaths. One of the stacking openings 210 or the tips 206 of the needle sheaths forms a crush ring that is configured to deform as the needle sheath is positioned through the stacking opening. The crush rings are configured such that the trays can be stacked by alignment the tip 206 of each needle sheath of a top tray through the corresponding stacking opening of the bottom tray. The crush rings are configured to deform as the end of the needle sheath is positioned through the stacking opening, increasing the friction of the needle sheath and the stacking opening to retain two trays 220, 224 in a stacked relationship as shown in FIG. 11. FIGS. 18-20 illustrate the preloaded needles 222 positioned within sheaths.

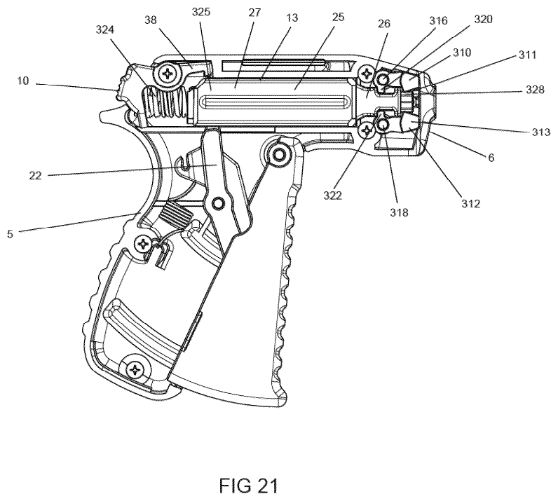

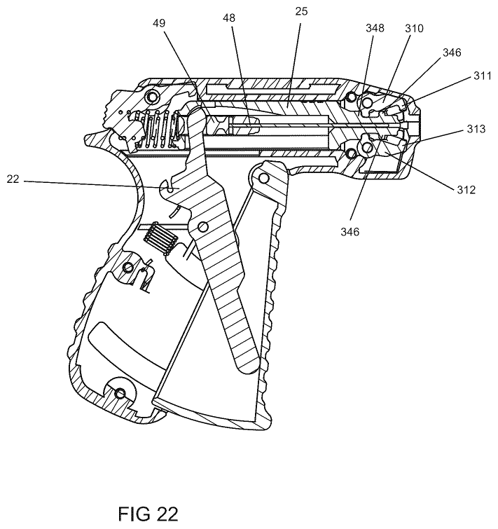

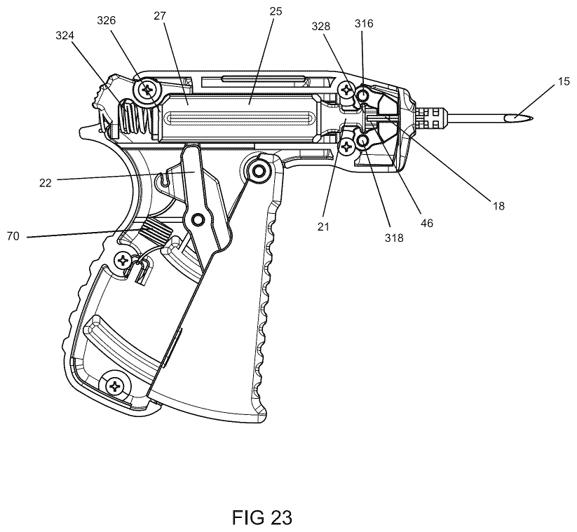

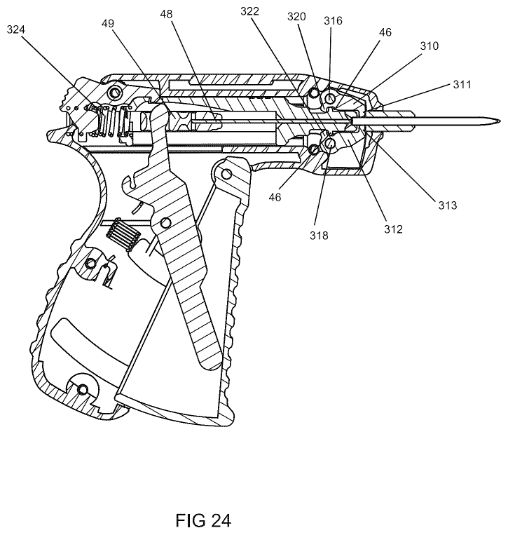

FIGS. 21-24 illustrate a preferred embodiment of an implanting gun. The implanting gun has a barrel 25 positioned within the gun housing 5. The barrel is configured to slide in the housing in a track formed within the housing in response to pressure from the compression spring 324 pressing the barrel forward, and back toward the hammer 10 from pressure from a needle being positioned in the muzzle of the gun onto the needle seat 328 of the barrel. The pressure overcomes the extension force of the spring, causing the barrel to slide toward the back of the gun. The barrel has a muzzle end 328 and a handle end 325. In the depicted embodiment the compression spring 324 extends from the rear of the gun housing to the handle end 325 of the barrel. The front 328 of the barrel is configured as a needle seat against which the needle base seats. The forward section of the barrel 26 has flanges 48 that engage with the tabs or pawls 320, 322. As the base of the needle is pushed against the front end of the barrel, the barrel 25 slides toward the rear of the housing, compressing spring 324.

The forward section of the barrel has a detent notch having a loading flange 362 and a release flange 348 that engage the detents 320, 322 of the opposing arms 312, 310 causing the arms to rotate on pivots 316, 318. This pivotal rotation causes the retaining hooks 311, 313 of the arms to engage the retaining flange 46 of the needle as the barrel is moved into the loaded or cocked position, and to release the retaining flange of the needle as the barrel moves forward.

To release the needle assembly, the hammer or tab 10 is depressed, disengaging the hook 38 of the hammer from a depression in the top 13 of the barrel. This allows the spring 324 to extend, causing the barrel to slide forward to release the hooks 310, 312 from the retaining flange 346. The release flange 348 of the detent notch engages the backside of the pawl causing the arms to rotate in the releasing direction opposite of when the barrel slides rearward.

As in the embodiment of FIG. 1, depression of the lever actuator 8 propels the push rod 48 through the barrel and expels the PIT tag from the needle. The end of the actuation lever travels in a slot configured in a bottom side of the barrel. In the depicted embodiment of FIGS. 21-24 the push rod is connected to a push rod seat 48. The end of the actuation lever engages the push rod seat to propel the push rod forward.

While certain preferred embodiments are shown in the figures and described in this disclosure, it is to be distinctly understood that the presently disclosed inventive concept(s) is not limited thereto but may be variously embodied to practice within the scope of the following claims. From the foregoing description, it will be apparent that various changes may be made without departing from the spirit and scope of the disclosure as defined by the following claims.

What is claimed is:

1. A handheld passive integrated transponder tag (PIT Tag) gun implanter and implanting needle combination comprising:
an implanter gun comprising:
a gun shaped housing having a barrel portion and a handle portion, said handle portion being disposed on said housing for being grasped by a hand of a user, said barrel portion extending to a muzzle, said gun housing having an opening at said muzzle configured to receive a needle hub;
a barrel positioned within said barrel portion, said barrel having a needle seat and a butt end;
a push rod housed at least partially in said barrel, wherein said push rod is configured to slide in said barrel;
a push rod actuator configured to slide said push rod in said barrel;
a releasable retention hook positioned in said barrel portion of said gun housing and configured to retain a retention flange of a needle; and
a retention hook trigger positioned at said rear end of said barrel portion and configured to release said retention hook from said retention flange; and
a needle comprising:
a needle hub releasably positioned in said barrel end of said gun housing against said needle seat;
a hollow steel rod comprising a beveled tip, said hollow steel rod extending from said needle hub and said barrel end of said housing, said needle hub comprising a retention flange secured by said retention hook;
a passive integrated transponder tag positioned within said needle; and
wherein said needle is positioned on said muzzle of said gun such that said needle is retained by said releasable retention hook, wherein said push rod actuator is configured to slide said push rod into said needle to eject said PIT Tag from tip of said needle.

2. The handheld PIT Tag implanter gun and implanting needle combination of claim 1 wherein said barrel is configured to slide in said barrel portion of said gun housing, wherein said retention hook trigger actuates sliding of said barrel to release said retention hook.

3. The handheld PIT Tag implanter gun and implanting needle combination of claim 2 further comprising a spring configured to bias said barrel forward, wherein said retention hook trigger retains said barrel with said spring in a compressed state, wherein releasing said retention hook trigger allows said spring to extend and causes said barrel to slide forward.

4. The handheld PIT Tag implanter gun and implanting needle combination of claim 2 comprising two retention hooks positioned on opposite sides of said barrel, wherein said retention hooks are pivotally connected to said housing, wherein each of said retention hooks comprises one of a detent or detent notch, wherein said barrel comprises the other of said detent or detent notch, wherein movement of said barrel forward and causes said barrel to pivotally rotate said retention hooks.

5. The handheld PIT Tag implanter gun and implanting needle combination of claim 1 where said barrel comprises an actuation flange at a rear end of said barrel, wherein said retention hook trigger comprises an actuation hook configured to engage said actuation flange, wherein depressing said retention hook trigger releases said actuation hook from said actuation flange.

6. The handheld PIT Tag implanter gun and implanting needle combination of claim 5 wherein said retention hook is rotatably connected to said barrel portion such that said barrel moving forward in said barrel portion of said housing rotates said retention hook out of engagement with said retention flange of said needle.

7. The handheld PIT Tag implanter gun and implanting needle combination of claim 3 wherein said barrel is configured such that insertion of a needle hub into said barrel end of said gun slides said barrel toward said rear of said barrel portion and engaging said actuation hook and causing said retention hook engage with said retention flange of said needle.

8. The handheld PIT Tag implanter gun and implanting needle combination of claim 1 wherein said barrel end of said housing comprises two opposing alignment slots, wherein said needle hub comprises two alignment tabs positioned on opposite sides of said needle base and configured for alignment within said alignment slots when said needle is loaded onto said muzzle.

9. The handheld PIT Tag implanter gun and implanting needle combination of claim 4 wherein said retention hook comprises a detent wheel, wherein an external circumference of said barrel comprises at least one detent notch configured to rotate said detent wheel when said barrel slides in said barrel portion of said housing.

10. The handheld PIT Tag implanter gun and implanting needle combination of claim 1 wherein said retention hook trigger is a depressible tab.

11. The handheld PIT Tag implanter gun and implanting needle combination of claim wherein said push rod actuator is in a handle portion.

12. The handheld PIT Tag implanter gun and implanting needle combination of claim 1 wherein said push rod actuator comprises a lever, wherein said lever slides in a slot in said barrel to slide said push rod.

13. The handheld PIT Tag implanter gun and implanting needle combination of claim 3 wherein said spring is configured to extend between an end of the barrel and the housing of the implanter gun.

14. The handheld PIT Tag implanter gun and implanting needle combination of claim 3 wherein said spring is positioned between the ends of said barrel.

15. An improved tray system for delivery of a plurality of needles pre-loaded with PIT Tags, said tray system comprising:
A pair of mating trays, each tray comprising a tray top having a plurality of needle sheath openings configured to receive a needle pre-loaded with a PIT Tag in a needle sheath extending beneath said tray top, said needle sheaths extending to a needle sheath tip, and a plurality of storage openings configured to receive said needle sheath tips such that said mating trays are in a stacked arrangement.

16. The improved tray system of claim 6 wherein one of said needle sheaths and said storage openings comprise a crush ring configured to deform to increase friction between said needle sheaths and said storage openings.

17. A needle for use with a PIT Tag implanter gun, said needle comprising:
a cylindrical hub configured for mounting in a muzzle of a PIT Tag implanter gun, said hub comprising a cylindrical conduit therethrough, said hub comprising an external surface having a retention flange and two opposing alignment tabs, said alignment tabs being configured to engage two opposing alignment slots in the PIT Tag implanter gun;
a hollow steel tube defining a lumen, said hollow steel tube extending from said cylindrical hub and coaxial with or continuous with said cylindrical conduit of said cylindrical hub such that a push rod in said implanter gun extends into said needle to expel a PIT Tag from said needle through a tip of said hollow steel tube, said hollow steel tube comprising a bevel; and
wherein said positioning tabs align said bevel to extend to be orientated to a side of the implanter gun.
